(19) United States
(12) Patent Application Publication
(10) Pub. No.: US 2022/0390742 A1
(43) Pub. Date: Dec. 8, 2022

(54) ANTI-FOG DEVICE HAVING LIQUID LENS ASSEMBLY WITH SPACE ISOLATED BY THIN FILM AND LENS INCLUDING THE SAME

(71) Applicant: ChaeHyun Lim, Seoul (KR)

(72) Inventor: ChaeHyun Lim, Seoul (KR)

(21) Appl. No.: 17/388,391

(22) Filed: Jul. 29, 2021

(30) Foreign Application Priority Data
Jun. 3, 2021 (KR) 10-2021-0071902

(51) Int. Cl. G02B 27/00; G02B 3/12; H05B 3/86

(52) U.S. Cl. CPC G02B 27/0006; G02B 3/12; H05B 3/86; A61F 9/029

(57) ABSTRACT

The present invention relates to an anti-fog device including: a lens unit including a first lens positioned outside and a second lens spaced apart from the first lens while facing the first lens to form an internal space and having a liquid filled in the internal space; a temperature control unit controlling a temperature of the liquid; and a circulation unit of which one side is connected to the internal space and the other side is connected to the temperature control unit to form a passage through which the liquid circulates. According to the present invention, it is possible to prevent record deterioration of a sports event or an accident during exercise by preventing occurrence of fog of goggles or swimming goggles to secure a visual field.

### CROSS-REFERENCE TO RELATED APPLICATION(S)

This application claims benefit of priority to Korean Patent Application No. 10-2021-0071902 filed on Jun. 3, 2021 in the Korean Intellectual Property Office, the disclosure of which is incorporated herein by reference in its entirety.

### BACKGROUND

### 1. Field

The present invention relates to an anti-fog device that prevents a dew condensation phenomenon of goggles, swimming goggles or the like.

### 2. Description of Related Art

In general, when a temperature of the air containing water vapor is cooled to the dew point or less, a dew condensation phenomenon in which water vapor contained in the air is condensed in the form of water droplets on a surface of an object occurs.

The dew condensation phenomenon occurring by an instantaneous temperature change mainly occurs by heating facilities in winter or air conditioners in summer, but may also occur due to a difference between a person's body temperature and an external temperature.

In particular, in a case where the person's body temperature rises as in sports, a difference between the body temperature and the external temperature may become larger, and accordingly, the dew condensation phenomenon also occurs on a surface of an object worn on a human body due to the difference between the body temperature and the external temperature.

For example, in a case of goggles or swimming goggles worn by athletes in order to secure a visual field in an sports event, fog such as the dew condensation phenomenon may also occur on inner surfaces of spaces formed between eyes to which the person's body temperature is transferred and the outside.

In particular, in a case of sports or leisure sports played underwater such as swimming or scuba diving, because of a difference between a body temperature raised during exercise and a relatively low temperature of a swimming pool or seawater and a large amount of moisture contained in the air around lenses of the swimming goggles due to characteristics of the swimming pool, fog may more severely occur.

In a case where the fog occurs on a surface of the lens, it obstructs a user's visual field and increases a risk of record deterioration or a safety accident.

Therefore, there is a need to prevent the occurrence of the fog in equipment that should be necessarily worn by a person for sports, such as the swimming goggles or the goggles, and a method capable of more conveniently secure the visual field without impairing an original purpose of the sports is required.

### SUMMARY

An object of the present invention is to propose an anti-fog device capable of preventing fog of goggles, swimming goggles or the like in order to reduce inconvenience caused by the fog.

More specifically, an object of the present invention is to propose a method of preventing fog through a natural physical phenomenon occurring in a state in which an original purpose of sports equipment is not limited.

Another object of the present invention is to propose a structure specialized for various auxiliary tools worn by a person in order to secure a visual field or make up for eyesight.

According to an aspect of the present invention, an anti-fog device may include: a lens unit including a first lens positioned outside and a second lens spaced apart from the first lens while facing the first lens to form an internal space and having a liquid filled in the internal space; a temperature control unit controlling a temperature of the liquid; and a circulation unit of which one side is connected to the internal space and the other side is connected to the temperature control unit to form a passage through which the liquid circulates.

The temperature control unit may include: a first temperature control unit controlling the temperature of the liquid using a heating coil connected to a battery; and a second temperature control unit in contact with a part of a user's body and controlling the temperature of the liquid using a user's body temperature.

The temperature control unit may selectively use the first temperature control unit and the second temperature control unit on the basis of the user's body temperature.

A correction film for correcting a refractive index by the liquid may be attached to an inner surface of the second lens.

The anti-fog device may further include a division film unit positioned in the internal space of the lens unit so as to be symmetrical with the first lens and the second lens and dividing the liquid.

The division film unit may be detachably positioned in the internal space, and be formed of a lens having a prescription.

### BRIEF DESCRIPTION OF DRAWINGS

The above and other aspects, features and other advantages of the present invention will be more clearly understood from the following detailed description taken in conjunction with the accompanying drawings, in which:

FIG. 1 is an illustrative view illustrating an anti-fog device according to an exemplary embodiment of the present invention;

FIGS. 2A and 2B are block diagrams illustrating components of the anti-fog device according to an exemplary embodiment of the present invention;

FIGS. 3 and 4 are illustrative views illustrating a lens unit of the anti-fog device according to an exemplary embodiment of the present invention;

FIG. 5 is an illustrative view illustrating the anti-fog device according to an exemplary embodiment of the present invention;

FIG. 6 is an illustrative view illustrating swimming goggles to which the anti-fog device according to an exemplary embodiment of the present invention is applied; and

FIG. 7 is an illustrative view illustrating goggles to which the anti-fog device according to an exemplary embodiment of the present invention is applied.

### DETAILED DESCRIPTION

The following description exemplifies only a principle of the present invention. Therefore, those skilled in the art may implement the principle of the present invention and invent various apparatuses included in the spirit and scope of the present invention although not clearly described or illustrated in the present specification. In addition, it is to be understood that all conditional terms and exemplary embodiments mentioned in the present specification are obviously intended only to allow those skilled in the art to understand a concept of the present invention in principle, and the present invention is not limited to exemplary embodiments and states particularly mentioned as such.

The objects, features, and advantages described above will become more obvious from the following detailed description provided in relation to the accompanying drawings. Therefore, those skilled in the art to which the present invention pertains may easily practice the technical spirit of the present invention.

Further, in describing the present invention, in the case in which it is determined that a detailed description of the well-known technology related to the present invention may unnecessarily make the gist of the present invention unclear, it will be omitted. Hereinafter, exemplary embodiments of the present invention will be described in detail with reference to the accompanying drawings.

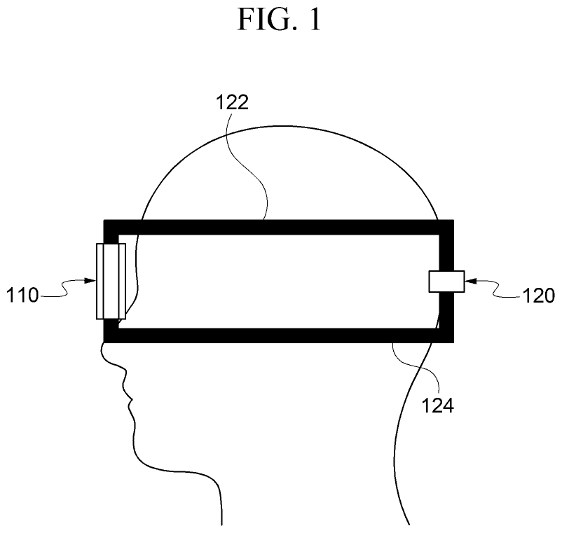

FIG. 1 is an illustrative view illustrating an anti-fog device through a liquid lens assembly having a space divided by a thin film according to an exemplary embodiment of the present invention.

Referring to FIG. 1, an anti-fog device 100 may include a lens unit 110, a temperature control unit 130, and a circulation unit 120, and may be detachably attached to goggles, swimming goggles or the like, or be formed as a partial component that adopts a physical structure of an existing component.

For example, the lens unit 110 may be configured in a form in which it is attachable to lenses of general swimming goggles, and the temperature control unit 130 may be formed on a band for wearing the swimming goggles. Therefore, a function according to the present invention may be implemented by wearing the band instead of an existing band.

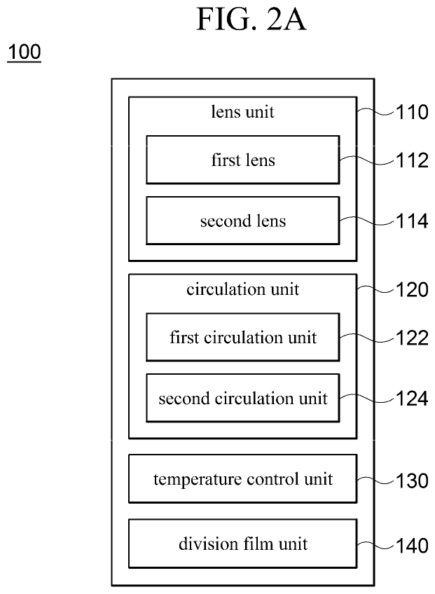

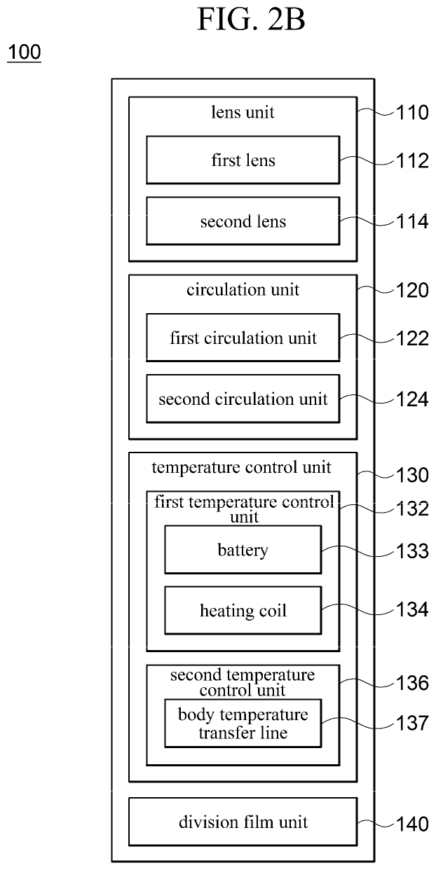

Specifically, components of the anti-fog device 100 according to the present embodiment will be described in more detail with reference to FIGS. 2A and 3.

The temperature control unit 130 of the anti-fog device 100 according to the present embodiment prevents fog by configuring the lenses doubly and controlling a temperature difference between the outside and the inside of the lenses using a liquid filled in a space between the lenses.

First, referring to FIG. 2A, the lens unit 110 of the anti-fog device 100 may include a first lens 112 positioned on an outer side on the basis of a wearer's body and in direct contact with the outside (such as water or air) and a second lens 114 spaced apart from the first lens 112 while facing the first lens 112 to form an internal space 31.

A liquid may be filled in the internal space 31, which is a free space formed by a gap between the first lens 112 and the second lens 114.

The liquid is purified water or is generally a colorless and transparent liquid, and may be injected using a component such as silicone oil having excellent flame resistance, cold resistance, water resistance, and chemical resistance. Specifically, the liquid may be injected into the internal space 31 of the lens unit 110 with a slight free space so as to be able to accommodate a volume change according to a temperature of the liquid, and may be filled, for example, to about 97% of the internal space 31. Therefore, it is possible to prevent air bubbles from being generated in the liquid due to excessive shaking during exercise, and it is possible to prevent damage to the internal space 31 due to the volume change according to the temperature of the liquid.

In addition, a partition wall between the lenses forming the internal space 31 may include an injection hole (not illustrated) through which the liquid may be injected and discharged for the continuous use of the anti-fog device 110. Therefore, it is possible to solve restriction of a visual field due to contamination of the liquid and allow the anti-fog device 20 to be permanently used.

In addition, referring to FIG. 3, a correction film for correcting distortion due to a difference in refractive index between the liquid and the first lens 112 or the second lens 114 may be further attached to a surface 33 of the second lens 114 in contact with the internal space 31 or a surface 35 of the first lens 112 in contact with the internal space 31.

The circulation unit 120 may be configured in the form of a band for wearing the swimming goggles or the goggles, one side of the circulation unit 120 may be directly connected to the internal space 31 of the lens unit 110, and the other side of the circulation unit 120 may be connected to the temperature control unit 130.

That is, a circulation structure of the lens unit 110 and the temperature control unit 130 formed by the circulation unit 120 performs a function of forming a flow passage so that the liquid in the internal space 31 circulates between the lens unit 110 and the temperature control unit 130.

Specifically, the circulation unit 120 may be divided horizontally or vertically in a state in which the anti-fog device 100 according to the present embodiment is worn, and may include, for example, referring to FIG. 1, a first circulation unit 122 and a second circulation unit 124 with the temperature control unit 136 as the center. In this case, the first circulation unit 122 may be configured to connect an upper end of the lens unit 110 and an upper end of the temperature control unit 130 to each other.

In addition, the second circulation unit 124 may be configured to connect a lower end of the lens unit 110 and a lower end side of the temperature control unit 130 to each other.

Therefore, it is possible implement a circulation structure in which the liquid whose temperature is lowered may move to the temperature control unit 130 through the second circulation unit 122 and the liquid heated in the temperature control unit 130 may move to the internal space 31 of the lens unit 110 through the first circulation unit 122.

That is, the anti-fog device 100 may circulate the liquid from the first circulation unit 122 toward the second circulation unit 124 through a convection phenomenon of the liquid without an external circulation device provided as a separate power device.

Furthermore, the temperature control unit 130 may include subsidiary structures such as a micro-valve for preventing backflow of the liquid moving through each circulation unit.

That is, the anti-fog device 100 may solve a problem such as contamination or air bubble generation caused by stagnation of the liquid only in the internal space of the lens unit 110 by continuously circulating the liquid in a predetermined direction through the first circulation unit 122 and the second circulation unit 124.

As described above, in the anti-fog device 100 according to the present embodiment, electric power may be reduced without installing an external power source or an electric wiring structure consumed in a separate circulation device, and an internal configuration may be further simplified.

In addition, by increasing a total mass of the liquid through the circulation unit 120 formed utilizing a surface area of the band for wearing the swimming goggles or the goggles, it is possible to increase heat capacity and improve an anti-fog effect accordingly.

Next, the temperature control unit 130 of the anti-fog device 100 may be connected to the circulation unit 120 to perform a function of controlling the temperature of the liquid filled in the internal space 31 of the lens unit 110.

In this case, the temperature control unit 130 serves to raise the temperature of the liquid inside the internal space through a person's body temperature during exercise, and may thus be configured at a position where a lot of heat is generated, such as a person's occipital region in a case where a person wears the swimming goggles. In addition, the temperature control unit 130 may further increase efficiency of temperature control according to the body temperature by forming a fine pattern structure such as a body temperature transfer line in order to increase a contact area with a human body and using a material having high conductivity in a portion in contact with the human body.

However, in the present embodiment, in order to further increase a temperature control effect by the temperature control unit 130, in a case of swimming goggles for leisure sports in which a restriction on a volume or a weight of a device is relatively less, it is also possible to selectively provide a temperature control component through electric power.

That is, the temperature control unit 130 may be selectively configured to include a first temperature control unit 132 controlling the temperature of the liquid using a heating coil 134 connected to a battery 133 and a second temperature control unit 136 in contact with a part of a user's body and controlling the temperature of the liquid using a user's body temperature.

This will be described with further reference to FIG. 2B.

Specifically, the first temperature control unit 132 may operate the heating coil 134 using electric power of the battery and control the temperature of the liquid by directly using heat of the heating coil 134.

In addition, the second temperature control unit 136 may be configured in a structure in which the fine pattern structure such as the body temperature transfer line 137 as described above is in contact with a user's skin to provide heat generated in the human body to the liquid circulating in the internal space 31, thereby controlling the temperature of the liquid.

In this case, the temperature control unit 130 may more efficiently control the temperature of the liquid filled in the internal space 31 by selectively using the first temperature control unit 132 and the second temperature control unit 136 on the basis of the user's body temperature.

For example, in a case where the user's body temperature rises to a reference temperature or more due to an increase in urgent movement according to a progress situation of an athletic sports event or in a case where a temperature difference between the user's body temperature and a temperature of external water is a reference temperature or more, the temperature control unit 130 controls the temperature of the liquid using the second temperature control unit. To the contrary, in a case where an intensity of exercise is low or the user's body temperature immediately after the start of exercise is not suitable for temperature control, the temperature control unit 130 may directly control the temperature of the liquid through external electric power using the first temperature control unit 132.

That is, the temperature control unit 130 may control the temperature of the liquid by using the second temperature control unit 136 in a case where the user's body temperature is higher than the reference temperature and using the first temperature control unit 132 in a case where the user's body temperature is lower than the reference temperature. Alternatively, it is also possible to select an operation mode by relatively using the temperature difference between the user's body temperature and the temperature of the external water.

Alternatively, it is also possible to increase entire efficiency by selecting a mode according to an electric power state of the battery 133.

In addition, in a case where the temperature of the liquid falls to a specific temperature or less, the temperature control unit 130 may more quickly prevent the occurrence of the fog by increasing the temperature of the liquid using both the first temperature control unit 132 and the second temperature control unit 136.

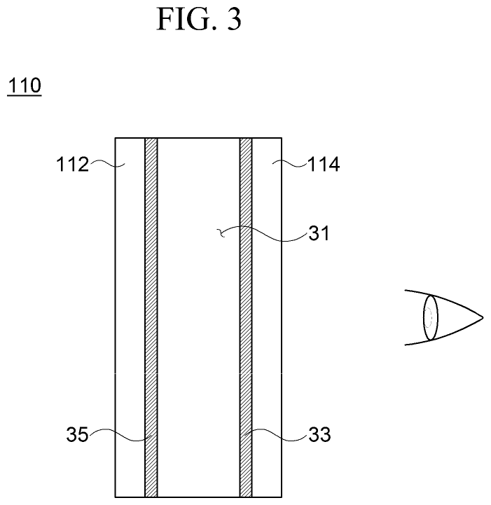

In addition, the anti-fog device 100 may further include a division film unit 140 in order to solve a subsidiary problem that may occur due to characteristics of the liquid itself. This will be described with reference to FIG. 4.

For example, in a case where the liquid itself in the internal space 31 is shaken by movement or a temperature of water of a swimming pool is 25° C. and the user's body temperature changes between 36.5° C. and 38° C., the temperature of the liquid in the internal space 31 may continuously change between 36.5° C. and 38° C. through the transfer of the user's body temperature. Therefore, a direct temperature difference occurs between a liquid of a front surface of the internal space 31 and a liquid of a rear surface of the internal space 31, and accordingly, a convection phenomenon may occur in the internal space 31 itself before circulation of the liquid through the circulation unit 120.

This phenomenon generates a problem of looking as if a haze continuously appears in water between the lenses due to a constant density difference.

Therefore, in order to solve such a problem, the anti-fog device 100 according to the present embodiment prevents a haze phenomenon by controlling a pressure of the liquid in the internal space 31 of the liquid to be a pressure for the movement of the circulation unit 120.

Referring to FIG. 4, the division film unit 140 of the anti-fog device 100 may be arranged to be parallel to the internal space 31 of the lens unit 110 formed by the first lens 112 and the second lens 114.

That is, the division film unit 140 may allow the liquid in the internal space 31 into a first space formed by the first lens 112 and the division film unit 140 and a second space formed by the division film unit 140 and the second lens 114.

Specifically, the division film unit 140 may divide the internal space 31 into a front space 21-1 and a rear space 21-2 on the basis of the division film unit 140, and allow the liquid to be divided into each of the front space 21-1 and the rear space 21-2. In this case, the division film unit 140 may allow the liquid to move in the spaces 21-1 and 21-2 divided through a micropore structure, and may induce convection due to a density difference to be first generated through the circulation unit 120 at a lower pressure. That is, even though a temperature difference occurs between the liquid in the front space 21-1 and the liquid in the rear space 21-2, the convection phenomenon occurring in the internal space 31 may be minimized.

As described above, the division film unit 140 may divide the liquid to minimize a phenomenon in which the liquid moves in the internal space 31 due to the movement of the user and minimize the movement of the liquid due to a density difference according to a temperature change of the liquid occurring when the liquid close to the outside cools first.

In addition, obstruction of a visual field (e.g., a phenomenon in which the liquid shimmers) due to the movement of the liquid may be minimized by minimizing the movement of the liquid.

In addition, one side of the circulation unit 120 may be configured to be separately connected to the front space 21-1 and the rear space 21-2 of the internal space 31, and the other side of the circulation unit 120 may be configured to be connected to the temperature control unit 130.

That is, the temperature control unit 130 may differently control the temperatures of the liquids in the front space 21-1 and the rear space 21-2.

In this case, the anti-fog device 100 may constantly maintain the temperature of the liquid by separately controlling the temperatures of the liquids in the front space 21-1 and the rear space 21-2 to minimize the temperature change of the liquid.

In addition, one division film unit 140 has been illustrated in FIG. 4, but a plurality of division film units 140 may be configured if necessary. In this case, the internal space 31 may be divided into several spaces, and the temperature of the liquid may be more precisely controlled.

Furthermore, the division film unit 140 may be implemented as a replaceable component that may be inserted into the space to be used appropriately depending on a situation in which the goggles or the swimming goggles are used.

Furthermore, the division film unit 140 may be formed of a lens having a prescription according to user's eyesight.

Therefore, the division film unit 140 may be formed of a lens, divide the internal space 31 of the lens unit 110 into the front space 21-1 and the rear space 21-2, and at the same time, provide eyesight correction to the user.

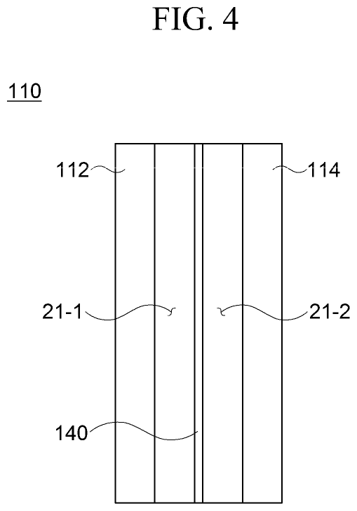

Next, an example in which a swimming cap 40 is coupled to the anti-fog device 100 will be described with reference to FIG. 5.

Referring to FIG. 5, the anti-fog device 100 may further include the swimming cap 40.

The circulation unit 120 of the anti-fog device 100 may also be implemented in the form of the swimming cap 40. That is, the swimming cap 40 may be configured by stacking two layers of surface, and the circulation unit 120 may be configured to transfer heat transferred through the user's body temperature from inner layer surface to the swimming goggles or the goggles. Therefore, the inner layer surface of the swimming cap 40 in contact with the user's skin is made of thinner surface to facilitate heat transfer, and outer layer surface of the swimming cap 40 in contact with the outside (e.g., the water of the swimming pool) is made of thick surface in order to implement a function of a swimming cap.

In addition, as in the above-described example implemented in the form of the swimming cap 40, the circulation unit 120 may include the first circulation unit 122 and the second circulation unit 124, and within the swimming cap 40, the first circulation unit 122 may be implemented to supply the heated liquid toward the lens and the second circulation unit 124 may be implemented to allow the liquid cooled by contact with the water to be again introduced into an inner layer of the swimming cap and heated.

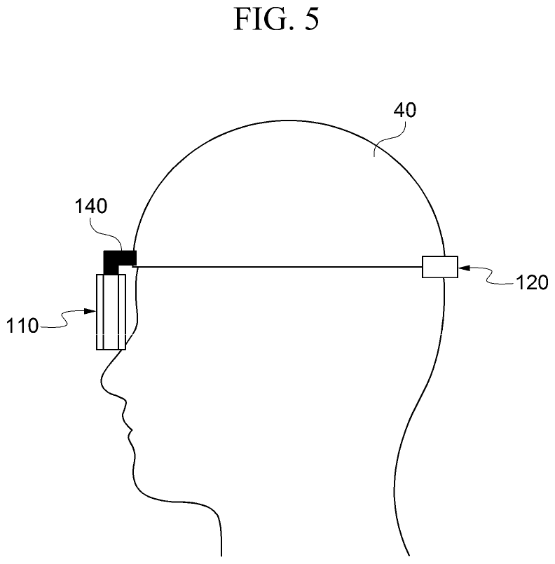

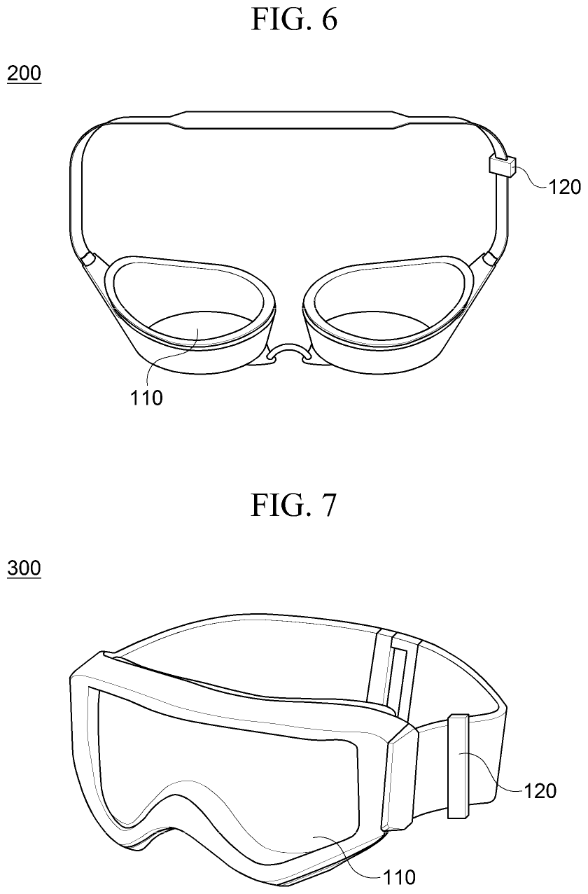

Next, examples in which the anti-fog device 100 is applied will be described with reference to FIGS. 6 and 7.

FIG. 6 is an illustrative view illustrating swimming goggles 200 to which the anti-fog device 100 according to an exemplary embodiment of the present invention is applied.

FIG. 7 is an illustrative view illustrating goggles 300 to which the anti-fog device 100 according to an exemplary embodiment of the present invention is applied.

First, referring to FIG. 6, the lens unit 110 of the anti-fog device 100 may be used as or attached to a front glass of the swimming goggles 200, and the temperature control unit 130 of the anti-fog device 100 may be attached to one side of a headband of the swimming goggles 200. In addition, the circulation unit 120 may be configured inside the headband.

In addition, referring to FIG. 7, the lens unit 110 of the anti-fog device 100 may be used as or attached to a front glass of the goggles 300, and the temperature control unit 130 of the anti-fog device 100 may be attached to one side of a headband of the goggles 300. In addition, the circulation unit 120 may be configured inside the headband.

As described above, according to the present invention, it is possible to prevent record deterioration of a sports event or an accident during exercise by preventing the occurrence of the fog of the goggles or the swimming goggles to secure the visual field.

In addition, according to the present invention, it is possible to minimize heterogeneity of a wearer by providing a structure capable of preventing the fog in a basic structure of the goggles or the swimming goggles and minimizing subsidiary components.

Furthermore, various exemplary embodiments described herein may be implemented in computer-readable recording medium using, for example, software, hardware, or a combination thereof.

According to a hardware implementation, exemplary embodiments described herein may be implemented using at least one of application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, and electric units for performing other functions. In some cases, exemplary embodiments described in the present specification may be implemented as a control module itself.

According to a software implementation, exemplary embodiments such as procedures and functions described in the present specification may be implemented as separate software modules. Each of the software modules may perform one or more functions and operations described in the present specification. A software code may be implemented as a software application written in a suitable programming language. The software code may be stored in a memory module and executed by a control module.

The technical spirit of the present invention has been described only by way of example hereinabove, and the present invention may be variously modified, altered, and substituted by those skilled in the art to which the present invention pertains without departing from essential features of the present invention.

Accordingly, exemplary embodiments disclosed in the present invention and the accompanying drawings are provided in order to describe the technical spirit of the present invention rather than limiting the technical spirit of the present invention, and the scope of the present invention is not limited by these exemplary embodiments and the accompanying drawings. The scope of the present invention should be interpreted by the following claims and it should be interpreted that all spirits equivalent to the following claims fall within the scope of the present invention.

What is claimed is:

1. An anti-fog device comprising:
a lens unit including a first lens positioned outside and a second lens spaced apart from the first lens while facing the first lens to form an internal space and having a liquid filled in the internal space;
a temperature control unit controlling a temperature of the liquid; and
a circulation unit of which one side is connected to the internal space and the other side is connected to the temperature control unit to form a passage through which the liquid circulates.

2. The anti-fog device of claim 1, wherein the temperature control unit includes:
a first temperature control unit controlling the temperature of the liquid using a heating coil connected to a battery; and
a second temperature control unit in contact with a part of a user's body and controlling the temperature of the liquid using a user's body temperature.

3. The anti-fog device of claim 2, wherein the temperature control unit selectively uses the first temperature control unit and the second temperature control unit on the basis of the user's body temperature.

4. The anti-fog device of claim 2, wherein a correction film for correcting a refractive index by the liquid is attached to an inner surface of the second lens.

5. The anti-fog device of claim 2, further comprising a division film unit positioned in the internal space of the lens unit so as to be symmetrical with the first lens and the second lens and dividing the liquid.

6. The anti-fog device of claim 5, wherein the division film unit is detachably positioned in the internal space, and is formed of a lens having a prescription.
